(19) United States
(12) Patent Application Publication
(10) Pub. No.: US 2008/0161779 A1
(43) Pub. Date: Jul. 3, 2008

(54) Implantable nano pump for drug delivery

(76) Inventor: Chiming Wei, (US)

Correspondence Address: Dr. Raj Bawa;Bawa Biotechnology Consulting LLC, 21005 Starflower Way, Ashburn, VA, 20147, US

(21) Appl. No.: 11/906,238

(22) Filed: Oct. 1, 2007

Related U.S. Application Data
(60) Provisional application No. 60/877,140, filed on Dec. 27, 2006.

(51) Int. Cl. A61M 5/00

(52) U.S. Cl. 604/891.1

(57) ABSTRACT

The present invention relates to nano-pumps (Nano Electromechanical System or NEMS), particularly to drug delivery devices for implantation into the human body for automatically control human diseases, such as hypertension, atherosclerosis and diabetics, etc. The nano-pump is self-actuated in response to a designated level of blood pressure, blood lipid or blood glucose in a human body without any other input source. Specifically, a flexible membrane acts as an integrated nano-sensor, nano-actuator and nano pump. Said membrane is circular in design and has a membrane pillar for stability and larger pumping force. The membrane is designed to compensate for the micro-viscosity effect in the pump. The pump has nano-valves composed of two portions, a semi-circle and a rectangle.

### CROSS-REFERENCE TO RELATED APPLICATION

This application claims benefit under 35 U.S.C. § 119(e) of U.S. provisional patent application No. 60/877,140, filed Dec. 27, 2006, the disclosure of which is herein incorporated in its entirety by reference.

### FIELD OF THE INVENTION

The present invention relates to nano-pumps (Nano Electromechanical System or NEMS), particularly to drug delivery devices for implantation into the human body for automatically control human diseases, such as hypertension, atherosclerosis and diabetics, etc.

### BACKGROUND OF THE INVENTION

An estimated 50 million Americans have hypertension, one of the major risk factors for stroke, coronary heart disease (CHD), heart failure, and other cardiovascular and renal diseases. Furthermore, atherosclerosis and diabetics are also the major risk factors in US for stroke, coronary heart disease, and heart failure. The effectiveness of detection, treatment, and control of hypertension, atherosclerosis and diabetics plays a major role in the primary and secondary prevention of these diseases. For stroke and heart failure, the relationship between blood pressure and risk is stronger than the relationship between blood pressure and coronary heart disease. The marked acceleration of the downward trend in age-adjusted stroke mortality in the United States after 1972 coincided with the formation of the National High Blood Pressure Education Program, a major national health education effort of the National Heart, lung, and Blood Institute of the National Institutes of health to detect, treat, and control hypertension.

In the United States, the prevalence of hypertension in adults is estimated at approximately 1 in 5 or 50 million persons. Each year, there are approximately 1 million new cases. Tens of millions of others have blood pressure (BP) levels above optimal, although not hypertensive (Table 1), and these persons are at increased risk of cardiovascular and renal diseases. As large as this blood pressure problem is overall, it is even more severe among those of lower socio-economic status, regardless of race and ethnicity.

Table 1 demonstrates the systolic blood pressure (SBP) and diastolic blood pressure (DBP) criteria for classification of blood pressure (BP) as optimal, normal (not optimal), high-normal, and high for adults age 18 years or older. The recommendation is to classify on the basis of average BP for an individual from ≧2 readings at each of ≧2 visits after an initial screening, with the individual not taking drugs and not acutely ill. Optimal BP, with regard to cardiovascular risk, is systolic blood pressure <120 mmHg and diastolic blood pressure <80 mmHg; however, unusually low readings should be evaluated for clinical significance. When systolic and diastolic blood pressures fall into different categories, the higher category should be selected to classify the individual BP status. (Based on the Fifth Report of the Joint National Committee on Detection, Evaluation, and Treatment of High Blood Pressure; National High Blood Pressure Education Program, National Institutes of Health, National heart, Lung, and Blood Institute, Bethesda, Md.: the Institute, 1995, NIH 93-1088).

TABLE 1

| | | Average Systolic | | |
|---|---|---|---|---|
| Average DBP | | Blood Pressure (mmHg) | | |
| (mmHg) | <120 | 120-129 | 130-139 | ≧140 |
| <80 | Optimal | Normal | High Normal | High |
| 80-84 | Normal | Normal | High Normal | High |
| 85-89 | High Normal | High Normal | High Normal | High |
| ≧90 | High | High | High | High |

Until the 1990s, the approach to coping with this mass blood pressure problem was primarily a “high-risk” strategy: detect, evaluate, and treat people with hypertension. This emphasis has accomplished much; it ended therapeutic nihilism in regard to hypertension and has resulted in control of hypertension for millions of Americans. It is a reasonable inference that this effort has been one of the most important factors contributing to the decades-long substantial declines in mortality rates from coronary heart disease and stroke and consequent increases in life expectancy for adult men and women.

But this high-risk strategy has serious limitations. It is late (i.e., defensive rather than proactive), and it relies primarily on drug treatment, with its mix of favorable and unfavorable effects and costs. In addition, millions of Americans with hypertension are treated inadequately or not at all, and tens of millions of other people with nonhypertensive blood pressure elevations are neglected despite their increased cardiovascular-renal risk. Above all, this high-risk strategy is never-ending; it offers no possibility of terminating the epidemic of high blood pressure. Only the primary prevention of this major risk factor offers this possibility.

A pivotal fact is that adverse blood pressure levels result from the rise in systolic and diastolic pressure experienced by most people during the decades from youth through middle age, with a continuing rise in systolic pressure through later years.

FIG. 1 shows mean systolic and diastolic blood pressures by age and race/ethnicity for men and women, U.S. population ≧18 year old. (From Burt V L, Whelton P, Roccella E J, et al. Prevalence of hypertension in the US adult population: results from the Third National health and Nutrition Examination Survey, 1998-1991, Hypertension. 1995; 25:305-313).

Lifelong maintenance of favorable blood pressure levels common among young adults would end high blood pressure as a mass problem. Recent research advances make this strategic goal possible.

FIG. 2 shows hypertension awareness, treatment, and control rates reported in the National health and Nutrition Examination Survey; age 18 to 74 years.

For persons between 18 and 74 years old, FIG. 2 shows marked improvement since 1971 to 1974 in the proportion of individuals with hypertension who are aware of their condition and being treated have their blood pressure under control. For these analyses, hypertension was defined by a blood pressure >160/95 mmHg or the use of anti hypertensive medication to include older data. Comparable percentages (numbers inside the bars) are available only for the 1976-1980 and 1988-1994 periods for the blood pressure threshold >140 mmHg systolic or >90 mmHg diastolic or those taking anti hypertensive medication.

During the NHANES III survey, which was conducted in 2 phases (1988-1991 and 1991-1994), there were small declines in awareness (73% to 68%), therapy (55% to 54%), and control (29% to 27%) of hypertension based on 140/90 mmHg cut points in persons 18 to 74 years of age. Preliminary data from the NHANES IV (1999-2000) show improvement from 1988 to 1994 in awareness, treatment, and control in men but no change in women. These survey data collected over 4 separate periods document the progress made during the 3 decades of national and community hypertension control efforts. These programs have alerted the public to the dangers of uncontrolled hypertension and its sequel and have successfully encouraged the public to visit their physicians, have blood pressures measured, follow their doctors' advice, and stay on therapy. Of concern is the recent report by the Surgeon General that 61% of Americans are overweight; accordingly, further improvements in hypertension control may be slower than in previous decades.

Evidence is mounting that treatment of those with isolated systolic hypertension and of persons with high-normal blood pressure levels is highly beneficial. An estimated 34 million Americans have high-normal blood pressure levels, 50 million Americans have hypertension, and approximately half of the population of hypertensives (approximately 25 million) has isolated systolic hypertension. Hypertension remains the major population-attributable risk factor for heart failure, the prevalence of which is approaching 5 million. Therefore, the control and treatment of hypertension is a very critical project in public health and will decrease unacceptable mortality and morbidity rates of cardiovascular diseases.

New emerging technologies (such as “Nano Electromechanical Systems” or NEMS) and materials (such as biocompatible polymers) enable the development of new drug delivery devices to improve medical treatments and patients' quality of life. Development of NEMS microfluidic device, such as nanopump, is a multidisciplinary research and development efforts, which covers a very broad range of studies including design, modeling, simulation, characterization, fabrication, micro flow pattern and phenomenon, etc. Recent development and commercialization of microfluidic devices were discussed in a review paper.

Anti-hypertensive, anti-atherosclerosis and anti-diabetics drugs are usually delivered after having detected the elevated blood pressure, blood lipid or blood glucose through irregular monitoring of the blood pressure blood sample. It is vital to develop a MEMS-based, miniaturized anti-hypertension, anti-atherosclerosis and anti-diabetics drug delivery device which responds in real-time to the rise of blood pressure, blood lipid or blood glucose at a designated level.

The very first documented reports of the concept of a micro pump, or miniaturized pump to be exact, could be found in U.S. Pat. No. 3,963,380 (Thomas, Jr. et al.) titled “Micro pump powered by piezoelectric disk benders”.

A second U.S. Pat. No. 4,152,098 (Moody et al.) is titled “Micropump”.

In the early 1980s, Stanford University initiated the research on micro pumps based on integrated chip (IC) micro fabrication techniques. These were piezoelectric type peristaltic micro pumps with three active valves and flow rate approximately 100 μl/min at 80V driving voltage and zero back pressure.

U.S. Pat. No. 5,171,132 (Miyazaki et al.) titled “Two-valve thin plate micropump” describes a micropump with a thin membrane plate.

U.S. Pat. No. 5,259,737 (Kamisuki et al.) titled “Micropump with valve structure” describes a micropump with a membrane and includes a diaphragm responsive to external pressure.

U.S. Pat. No. 6,986,649 (Dai et al.) is titled “Micropump with integrated pressure sensor”.

However these prior art do not describe a self-actuated micro pump with integrated micro-sensor, micro-actuator and micro pump.

### SUMMARY OF THE INVENTION

### Objects of the Invention

It is therefore an object of this invention to provide a micro pump or nanopump, implantable in a human body, which is self-actuated in response to a designated level of blood pressure, blood lipid or blood glucose in a human body without any other input source.

Another object of this invention to provide a device with integrated micro-sensor, micro-actuator and micro pump.

Another object of this invention to provide a micro pump with a membrane and a membrane pillar which generates a larger pumping force enhances stability of the downward deflection of the membrane and prolongs the life cycle of the membrane.

Another object of this invention to provide a micro pump with a membrane which has a circular design which ensures that the stresses exerted on the membrane and the sidewall of the micro chamber of the micro pump are uniformly distributed.

Another object of this invention is to provide a linked array or a 2-D matrix of nanopumps for larger dose requirements.

Another object of this invention is to provide a group of nanopumps having individual nanopumps which are designed to respond to different blood pressure, blood lipid or blood glucose levels by having membranes of varying thickness in the individual nanopumps, thus enabling multi-level blood pressure drug release.

Another object of this invention is to reckon and compensate for the micro-viscosity effect in the micro pump.

Another object of this invention is to provide a new nano-valve for the systolic nanopump wherein the nano-valve is composed of two portions, a semi-circle and a rectangle.

In the prior art, micro pumps are generally activated by different types of micro actuators with high driving frequencies. They are not suitable for hypertension treatment. A real-time drug delivery micro pump implantable for hypertension treatment must be miniaturized and self-actuated in response to elevated blood pressure. Hence, the present invention has been developed with specific application to hypertension treatment.

### Significance

Anti-hypertensive, anti-atherosclerosis and anti-diabetics drugs are usually delivered after having detected the elevated blood pressure, blood lipid or blood glucose levels through irregular monitoring of the blood pressure, blood lipid or blood glucose. It is vital to develop a MEMS-based, miniaturized anti-hypertension, anti-atherosclerosis and anti-diabetics drug delivery device which responds in real-time to the rise of blood pressure, blood lipid or blood glucose at a designated level. The present invention is an innovative system for the treatment and control of hypertension, atherosclerosis and diabetics. It is a self-actuated NEMS-based smart micro pump with bossed membrane (SM/BM). It could significantly decrease the adverse morbidity and mortality rates associated with hypertension, diabetes, and atherosclerosis related cardiovascular diseases.

Unlike other micro pumps which are activated by micro actuators, the nanopump of the present invention is activated by elevated blood pressure, blood lipid or blood glucose level. Once the elevated blood pressure, blood lipid or blood glucose is sensed by the membrane, the membrane deflects and the drug originally stored in the nano-chamber is pumped out immediately.

### BRIEF DESCRIPTION OF THE DRAWINGS

While the specification concludes with claims particularly pointing out and distinctly claiming the subject matter that is regarded as the present invention, it is believed that a more complete understanding of the invention may be obtained by reading the following description in conjunction with the appended drawings in which:

FIG. 1 shows trends in mean systolic and diastolic blood pressures by age and race/ethnicity for men and women in the U.S. Population;

FIG. 2 shows trends in Hypertension awareness, treatment and control rates reported in the National Health and Nutrition Examination Survey, age 18 to 74 years;

FIG. 3 embodies a typical 4-layer (i.e. 4 wafers) integrated micropump of the present invention;

FIG. 4 embodies an external micropump;

FIG. 5 is a chart showing how the micro-viscosity of the fluid that contains anti-hypertension drugs will be analyzed;

FIG. 6 shows a conventional flat membrane design;

FIG. 7 shows a bossed membrane design;

FIG. 8 shows a new nano-valve design for the systolic nanopump; and

FIG. 9 is a chart showing how finite element analysis together with the membrane optimization algorithm and flow rate analysis to characterize the structural behavior of the entire systolic nanopump will be performed.

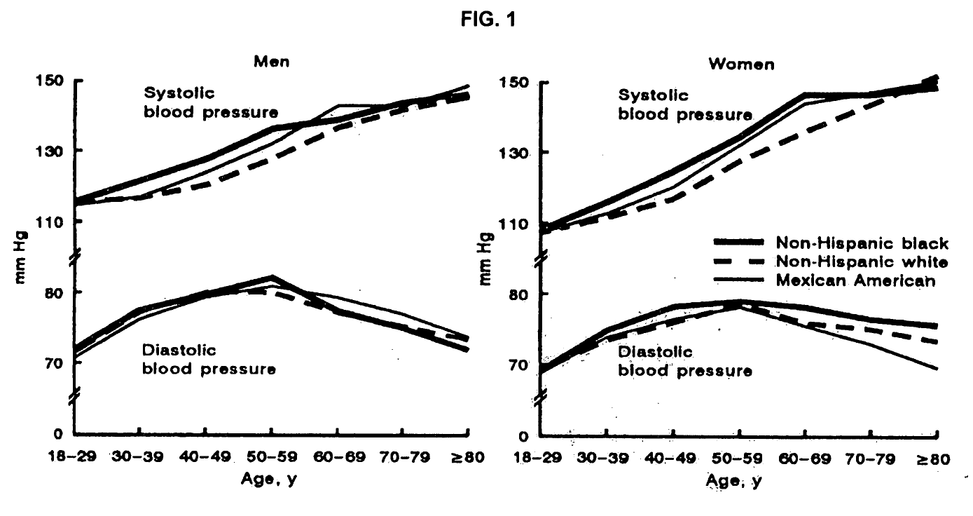

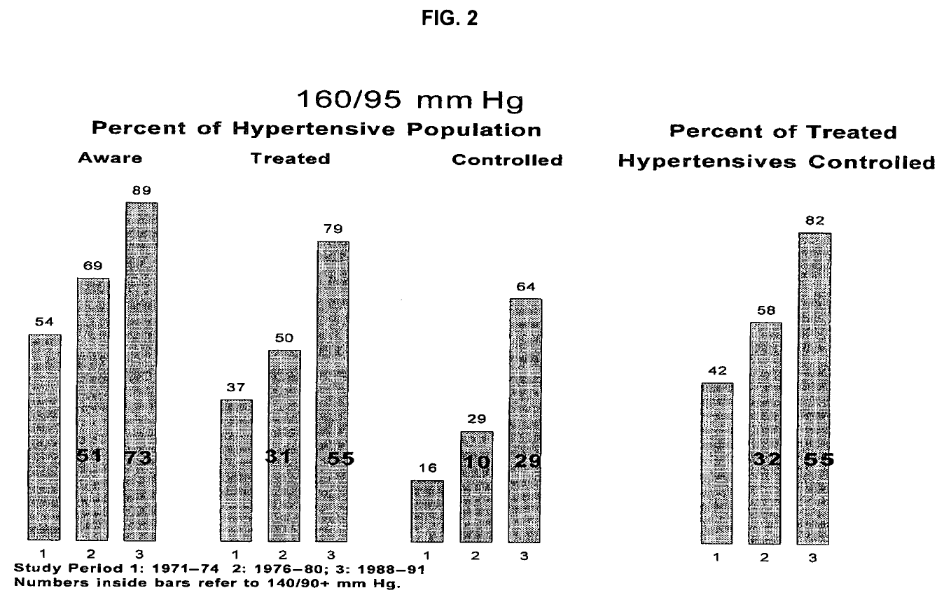

### DETAILED DESCRIPTION OF THE EMBODIMENTS OF THE INVENTION

In this specification, although the preferred embodiments have been described in detail, it should be understood that various changes, substitutions and alterations may be made therein without departing from the spirit and scope of the invention. Therefore, the specification is to be regarded in an illustrative rather than a restrictive sense. In other words, the described embodiments of the invention and the disclosed examples are given for the purpose of illustration rather than limitation of the invention as set forth in the appended claims.

Unless otherwise defined herein, scientific and technical terms used in connection with the present invention shall have the meanings that are commonly understood by those of ordinary skill in the art. In this disclosure, the terms “nanopump” and “micro pump” are used interchangeably.

Furthermore, unless otherwise required by context, singular terms shall include pluralities and plural terms shall include the singular. Generally, nomenclatures used in connection with, and standard techniques described herein are those well known and commonly used in the art. Furthermore, unless stated to the contrary, any use of the words such as “including,” “containing,” “comprising,” “having” and the like, means “including without limitation” and shall not be construed to limit any general statement that it follows to the specific or similar items or matters immediately following it. It should also be understood that the various embodiments of the present invention are not mutually exclusive, but may be implemented in various combinations.

The present invention is a self-actuated NEMS-based smart micro pump with bossed membrane (SM/BM). The quasi-static model under the assumption of low operating frequency is used to characterize SM/BM. The mathematical model is an ordinary differential equation which describes its behavior considering its key components—membrane and micro valves. To verify the range within which the quasi-static model is valid, the fundamental natural frequencies of bossed membrane and micro valves are investigated. The bossed membrane and micro valves are characterized by Finite Element Analysis (FEA) package ANSYS. The stroke volumes for the membrane are calculated within the range that the linear deflection theory is valid in. Analysis of micro valve is a challenging task in microfluidics because it is a coupled-field problem where solving the structural or fluid part separately is unachievable. Based on the sequential weak solid-fluid coupling in ANSYS/FLOTRAN, the flow rates across inlet and outlet micro valves are simulated.

Since the quasi-static equation contains non-linear terms, the analytical solution is impossible, and MATLAB® is used to solve it numerically. Its solution is based on membrane and micro valves characteristics for an assigned driving pressure yields chamber pressure. The transient flow of the pump is then obtained by substituting chamber pressure into the outlet micro valve flow rate function. Integrating the previous function over one cycle and then multiplying by the assigned frequency results in the pump flow rate. The highest flow rate is 75.8 μl/min for the design and conditions analyzed in this paper. The SM/BM transit behavior and back pressure effect on performance are shown.

### Micro Pumps and Nanopumps

According to different operating (i.e., actuating) principles, micro pumps are divided into mechanical micro pumps and non-mechanical micro pumps. Mechanical micro pumps are by far the most popular and widely studied, which includes reciprocating micro pumps, peristaltic micro pumps, and rotary micro pumps. Reciprocating micro pumps have been investigated more extensively than any other types of micro pumps. Zengerle attributes that to the possibility of pumping most fluids where no special demand on the electrical properties of the fluid or other significant restriction. The reciprocating micro pump can be further categorized by its actuation mechanism as follows: piezoelectric, electrostatic, thermo-pneumatic, pneumatic, shape memory alloy, bimetallic, and electromagnetic. The non-mechanical pumps have neither moving parts nor valves. Consequently, advantages include simplicity in design and fabrication, longer cyclic life and minimal risk of blockage. However, Non-mechanical micro pumps are feasible only on the micro scale as the forces generated by such means are generally unable to move larger quantities of fluids. Much fewer non-mechanical micro pumps have ever been fabricated and tested successfully. Non-mechanical micro pumps include electro-hydrodynamic (EHD) micro pump, electro-osmotic micro pump, ultrasonic micro pump, magnetic micro pump, and other types of non-mechanical micro pumps.

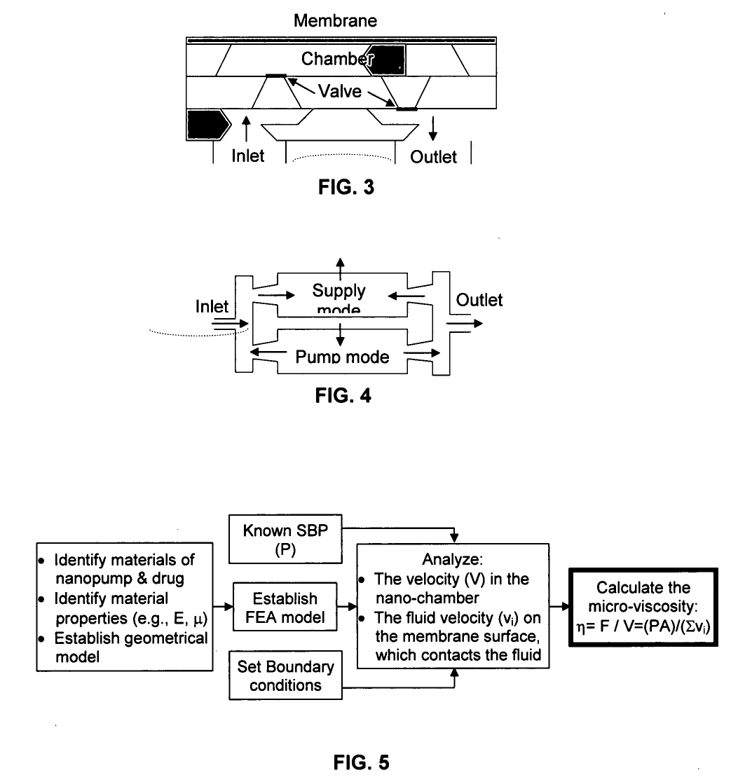

Based on different structures, there are two types of micro pumps: integrated and external. Integrated micro pumps utilize the membrane (i.e., diaphragm) vibration to produce pumping operation. FIG. 3 shows a typical 4-layer (i.e., 4 wafers) integrated micro pump. External micro pumps, FIG. 4, use inlet/outlet ports of the micro pump to connect to the micro-channel. Survey on the two active components—micro valve and membrane—is discussed hereinafter.

Micro valve is one of the most important components in the microfluidic systems. Different types of micro valves can be sorted in terms of being used for liquids or gases, whether normally open or closed, or digital or proportional. The more logical classification is based on their actuation mechanisms. In this case, micro valves are classified as passive and active valves. Passive micro valves are mainly used in micro pumps with mechanical actuation, and the micro pumps use these valves to direct the flow. There is no built-in actuating device in the valve; they are activated (e.g., pressed open or closed) by the fluid pressure. They are designed to provide a high flow rate in one direction (low fluid resistance) and low flow rate in the opposite direction (high fluid resistance). There are several different types of passive micro valves: cantilever micro valve, diaphragm micro valve, diffuser/nozzle micro valve, and bi-valvular micro valve. A wide range of actuation schemes are used for active micro valves, each one has its own advantages and drawbacks. The most commonly used actuation mechanisms for active micro valves are the same as those introduced above for reciprocating micro pumps.

Membrane (i.e., diaphragm) is another critical element in a micro pump. Researchers have put in great efforts to study the membrane of a micro pump and other MEMS devices. Analysis of membrane performance, design, deflections and stresses for flat membranes, flat membranes with central boss and corrugated membranes are known in the art. Membranes have linear and nonlinear operating regions. The bossed membrane structure offers better linearity (i.e., more predictable and controllable behavior of the membrane) than a non-bossed structure. Compared to the flat membranes, the corrugated membranes can increase the net deflection for equivalent loads and the linear operating range. These are considered the advantages of corrugated membranes. However, one of the major problems in fabricating the corrugated membranes is the internal stresses which affect the deflection behavior. The mechanical sensitivity (increase in deflection over the increase in applied pressure) for corrugated diaphragms with certain geometric parameters can be 25 times larger than the flat diaphragms of equal size and thickness. Moreover, it is more costly and difficult to fabricate the corrugated membranes. Consequently, our design focuses on the flat and bossed membranes only.

### Design of the Systolic Nanopump

The present invention has been designed specifically for hypertension, diabetes, and atherosclerosis treatment using optimization technique to maximize its efficiency (i.e., maximal drug delivery rate). The FEA (finite element analysis) is carried out to characterize its performance and structural behavior and to validate the analytical model.

(1) Working Principle

Since more than 60% of the hypertension patients are of high systolic blood pressure (>140 mmHG), this nanopump has been designed to react to the systolic blood pressure (SBP) elevation and deliver the anti-hypertension drug automatically in real-time. However, the technology developed for this “systolic nanopump” is completely useful for developing the second type of “diastolic nanopump.” Unlike other micro pumps which are activated by micro actuators, “systolic nanopump” is activated by the elevated SBP. Once the elevated SBP is sensed by the membrane, the membrane deflects and the drug originally stored in the nano-chamber is pumped out immediately. This has led to the basic conceptual design of the systolic nanopump. Moreover, the thicker the membrane, the stiffer it is; that is, the membrane will respond to a higher SBP level. On the other hand, if the membrane is thinner, it responds to lower SBP level. Therefore, the membrane thickness of a systolic nanopump must be precisely defined to be self-activated at a designated SBP level. These differentiate the systolic nanopump from the EMMP because the membrane thickness of an EMMP is not a main concern at all.

In addition to the innovative self-actuating design of the present systolic nanopump, other innovations include:
1. Integration of micro-sensor, micro-actuator and micro pump into one single unit—The systolic nanopump is self-actuated in real-time in response to a designated SBP level without any other input source. The membrane serves as a pressure sensor and, at the same time, a micro-actuator,
2. Membrane pillar—Most membrane designs are uniform in thickness; advantages of adding a membrane pillar are a) to generate larger pumping force, that is, larger drug delivery rate, b) to enhance stability (of the downward linear deflection) of the membrane resulted from the inertia of the pillar, and c) to prolong the life cycle of the membrane, that is, the life of the systolic micro pump, and
3. Circular membrane design—Once the membrane is actuated, there will be stresses exerted on the membrane and the sidewall of the micro-chamber. Most of the membrane designs are square or rectangular in shape; the advantage of using a circular membrane is that the stresses will be more uniformly distributed both on the membrane and on the circumference of the sidewall of the micro-chamber.

(2) Critical Design Components and Parameters

Nomenclatures of the critical parameters are defined in the Table 2 (next page). It explains that the systolic nanopump of the present invention will be fabricated in 3 wafers and its corresponding physical components are identified by independent design parameters. These parameters are to be used to further analyze, optimize, and characterize the systolic nanopump.

TABLE 2
Critical design components and parameters of the systolic nanopump

| Systolic | | | Diameter | |
|---|---|---|---|---|
| nanopump | Component | | (μm) | Thickness (μm) |
| Nano-sensor | Wafer 1 | Membrane | dₘ | tₘ |
| | | Membrane pillar | dₘₚ | tₘₚ |
| | | Nano-chamber | dₘ | t_{c} |
| | | Sidewall | dₗ | tₘ + t_{c} |
| Nano-pump | Wafer 2 | Inlet nano-channel | d_{mc} | t₂ |
| | | Inlet nano-valve | dᵢᵥ | tᵢᵥ |
| | Wafer 3 | Inlet nano-channel | dᵢ | t₃ |
| | | Outlet nano-channel | dₒ | t₃ |
| | | Outlet nano-valve | dₒᵥ | tₒᵥ |

d = distance from inlet nano-channel to the inner wall of the nano-chamber
d_{mc} = distance between nano-channels

In one embodiment of the invention, multiple systolic nanopumps are linked together to form an array or a 2-D matrix for larger dose requirements.

Another embodiment of the invention comprises a group of nanopumps having individual nanopumps which are designed to respond to different blood pressure, blood lipid or blood glucose levels by having membranes of varying thickness in the individual nanopumps. This enables multi-level blood pressure, blood lipid or blood glucose drug release wherein the medication and the quantity of medication are customized and responsive to different blood pressure levels or therapeutic treatment requirement levels.

(3) Characterization of Membrane

As consulted with experts in anti-hypertensive research, the maximum allowable diameter (d₁) of the implantable systolic nanopump should be within 1 mm (i.e., 1,000 μm). Owing to this, this objective was designed to test different sizes (100, 250, 500, 750 and 1,000 μm).

(4) Micro-Viscosity Effect

In analogy to the friction between solids, the viscosity is the “friction” between the fluid and its contacting solid surface (e.g., the membrane surface). It was brought to our attention during the preliminary study for EMMP that micro-viscosity plays a much more important role than is often considered. Based on our analysis, the control frequency of the membrane is 29.5 KHz for “dry” EMMP (i.e., no fluid in the micro-chamber); however, its control frequency drastically reduced to 15.9 KHz (˜46% reduction) when the micro-chamber contains water. This will affect the overall performance (e.g., drug delivery rate) of the EMMP. The author believes the control frequency was diminished by the viscosity effect, and the viscosity effect will be magnified when the size of the nanopump becomes smaller, especially in the micron rang.

Therefore, it is essential to reckon and compensate for the micro-viscosity effect in the present invention. The viscosity of fluid that contains drug can even be higher than that of water (10⁻⁶ Kg/mm.s). To deliver an exact amount of drug using any nanopump, the dose must be adjusted to compensate the loss caused by the viscosity effect. The micro-viscosity of the fluid that contains anti-hypertension drugs based on the mathematical model is analyzed as shown in FIG. 5.

The nano-viscosity η (N.s/m) is analyzed as a function of the SBP (P), membrane area (A), and the resultant velocity (V). The resultant velocity is the sum of the velocity components (Σvᵢ) distributed on the membrane surface that in contact with the fluid. The nano-viscosity (η) effect on the systolic micro pump as a function of membrane deflection will be analyzed, which varies with thickness. Also, the maximum flow rate (Qₘₐₓ) can be calculated as a function of membrane thickness and SBP. The average flow rate Q will then be adjusted by the micro-viscosity effect accordingly.

(5) Membrane Deflection

The membrane is one of the most important components of the systolic nanopump. The membrane material, diameter and thickness must be carefully analyzed to be actuated at a designated SBP level. Under this objective area, nano-viscosity effect on membrane deflection will be thoroughly analyzed and compared among the following four cases: 1) Flat membrane deflection without micro-viscosity effect, 2) Bossed membrane deflection without micro-viscosity effect, 3) Flat membrane deflection with nano-viscosity effect, and 4) Bossed membrane deflection with nano-viscosity effect. The study of the first 2 cases can be easily found in many literatures as described below. The last 2 cases proposed in this project that involve nano-viscosity effect will be brand new studies.

(a) Flat Membrane Deflection without Nano-Viscosity Effect

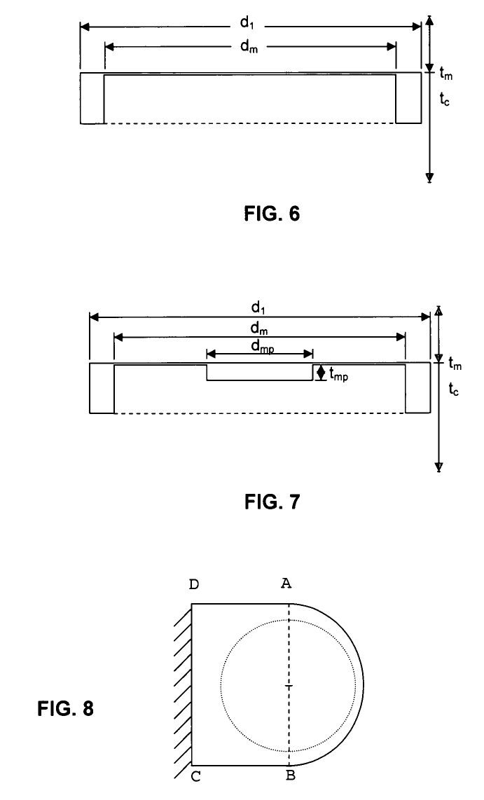

As mentioned earlier, conventional membrane design for micro pump is uniform in thickness as shown in FIG. 6, where dₗ is the diameter of the nanopump, dₘ is the diameter of the membrane, tₘ is the thickness of the membrane, and t_{c} is the thickness (or height) of the side-wall of the micro-chamber. The membrane deflection (y) can be calculated using the equation of a circular membrane:

$$\frac{{P ( {d}_{m} / 2 )}^{4}}{E {t}_{m}^{4}} = \frac{16}{3 ( 1 - {μ}^{2} )} \frac{y}{{t}_{m}} + \frac{7 - μ}{3 ( 1 - μ )} \frac{{y}^{3}}{{t}_{m}^{3}} \qquad ( 4 )$$

where y is the membrane deflection and solved by rearranging the equation. P is pressure (force/area), dₘ is the diameter of the membrane, E is the Young's modulus of the membrane, tₘ is thickness of the membrane, and μ is the Poisson's ratio (0.5 for PDMS). Equation (4) contains two deflection terms: bending

$$\frac{16}{3 ( 1 - {μ}^{2} )} \frac{y}{{t}_{m}}$$

and tension

$$\frac{7 - μ}{3 ( 1 - μ )} \frac{{y}^{3}}{{t}_{m}^{3}} ,$$

which applies the superposition method to add these two terms together.

(b) Bossed Membrane Deflection without Micro-Viscosity Effect

The novel design of membrane with membrane pillar is depicted in FIG. 7. Two more parameters are introduced: the diameter of the membrane pillar (dₘₚ) and the thickness of the membrane pillar (tₘₚ). To be effective, tₘₚ should be at least six times thicker than tₘ, that is, tₘₚ≧6tₘ. The solidity ratio (dₘₚ/dₘ) is a very important parameter in the performance characteristics of the membrane with pillar. The solidity ratio of less than 0.15 has little effect on the stiffness of the membrane. However, higher solidity ratio makes the membrane progressively stiffer. The characteristic equation to calculate deflection (y) of a circular bossed membrane is:

$$\frac{{P ( {d}_{m} / 2 )}^{4}}{E {t}_{m}^{4}} = \frac{1}{{A}_{p}} \frac{y}{{t}_{m}} + {B}_{p} \frac{{y}^{3}}{{t}_{m}^{3}} \qquad ( 5 )$$

where

$${A}_{p} = \frac{3 ( 1 - {μ}^{2} )}{16} ( 1 - \frac{{( {d}_{mp} / 2 )}^{4}}{{( {d}_{m} / 2 )}^{4}} - 4 \frac{{( {d}_{mp} / 2 )}^{2}}{{( {d}_{m} / 2 )}^{2}} log \frac{{d}_{m}}{{d}_{mp}} )$$

is the stiffness coefficient of the linear term, and

$${B}_{p} = \frac{\frac{7 - μ}{3} ( 1 + \frac{{( {d}_{mp} / 2 )}^{2}}{{( {d}_{m} / 2 )}^{2}} + \frac{{( {d}_{mp} / 2 )}^{4}}{{( {d}_{m} / 2 )}^{4}} ) + \frac{{( 3 - μ )}^{2}}{1 + μ} \frac{{( {d}_{mp} / 2 )}^{2}}{{( {d}_{m} / 2 )}^{2}}}{( 1 - μ ) ( 1 - \frac{{( {d}_{mp} / 2 )}^{4}}{{( {d}_{m} / 2 )}^{4}} ) {( 1 - \frac{{( {d}_{mp} / 2 )}^{2}}{{( {d}_{m} / 2 )}^{2}} )}^{2}}$$

is the stiffness coefficient for the nonlinear term.

(c) Membrane Deflection with Nano-Viscosity Effect

Study of nano-viscosity effect on membrane (both flat and bossed) deflection is novel. Since viscosity is the “shear” friction between the fluid and its contacting solid surface (i.e., the membrane surface), an additional shear (stress) term in the membrane deflection equation will be derived.

(6) Micro-Viscosity Effect on Flow Rate

Flow rate for each of the four cases mentioned above will be analyzed and compared to understand the micro-viscosity effect. As described in section C. Preliminary Studies, similar approaches using the ANSYS FEA will be employed.

(7) Optimization of Membrane

The systolic nanopump membrane design is further refined using the numerical design optimization technique. The optimization procedures were formulated as follows:

A design optimization problem is usually highly non-linear with multiple design variables and constraints. An optimization problem is generally formulated in the following mathematical form:

Solve for design variables (X₁, X₂, X₃, . . . , Xₙ)

To minimize the Objective Function F(X₁, X₂, X₃, . . . , Xₙ)

subject to constraints X_{i,min}≦X_{i,max} (i=1 to n)

with side constraints q_{i,min}≦q_{i,max} (j=1 to m)

In practice, the design variables can be any, or all, of the critical parameters defined in Table 2. Other parameters (e.g., material properties, desired pressure level, flow rate, etc.) can be added as additional design variables. The design constraint is the acceptable design range of a specified design variable within which the solution is sought. For example, to optimize the membrane thickness (tₘ), it would be appropriate to define a minimum/maximum range of tₘ (i.e., t_{m,min}≦tₘ≦t_{m,max}) as its design constraint. The values of t_{m,min} and t_{m,max} should be defined in a reasonable manner.

The optimization problem then is formulated as:

Solve for design variables (X₁, X₂, X₃, . . . , Xₙ)

To minimize the deflection objective function y (X₁, X₂, X₃, . . . Xₙ)

Subject to constraints X_{i,min}≦Xᵢ≦X_{,max} (i=1 to n)

With side constraint Q≦Qₘₐₓ

where y is the deflection objective function for membrane to be minimized. Equations derived for the four different cases mentioned in preceding section will be rewritten as the deflection objective function. If the amount of dosage (concentration administered in one injection) to be delivered for hypertension treatment is H_{d} ug, with known fluid-drug density ρ (ug/um³), the Q can be estimated as H_{d}/ρ after a duration, t (sec), of release. Once Qₘₐₓ is estimated, it quantifies the side constraint Q. Since Q is correlated to the flow velocity (V_{f}) and area of the outlet micro-channel (d₀), V_{f}, d₀, and drug release time (t) now can be assigned as constraints X₁, X₂, and X₃ along with individual range of minimum/maximum value. Another critical parameter for the membrane design is definitely the exact value (or range) of SBP, the project was assigned it as X₄ and let X₄=140 mmHg (or 140 mmHg≧X₄≦1500 mmHg). To this point, the project has defined 4 constraints {X₁, X₂, X₃, X₄}={V_{f}, d₀, t, SBP}. More constraints can be selected from any of the parameters defined in Table 2. Other appropriate constraints can also be added. The membrane for all four cases discussed in previous section will be optimized.

(8) Characterization of Cantilever Nano-Valve

In the above optimization approach, the value of flow rate (Q) is defined as a side constraint (Q≦Qₘₐₓ). In this section, the more detailed study of the effect of another critical component—nano-valve—on the flow rate will be performed. Nano-valve controls the fluid flow speed and flow rate and affects the overall performance of a nanopump. We will use the cantilever check-valves, which simplifies the fabrication and control process of the IMSP.

Almost all nano-valves are rectangular in shape. A new nano-valve is designed for the systolic nanopump as presented in FIG. 8. The nano-valve is composed of two portions: a semi-circle AB and a rectangle ABCD. The reason for this design is to have a better fit for the circular nano-channel shown as a dotted circle. Deflection and flow rate of the nano-valve with different dimensions relative to various flow velocities and sizes of micro-channels will be analyzed. Once the governing equations of deflection (δ), flow rate (Q) and SBP (P) for the new nano-valve are derived, Mathematica software package will be used to solve these integral equations. Analytical results will be further compared with conventional rectangular check-valve to evaluate the efficacy of the new nano-valve design.

### Characterization of Systolic Nanopump

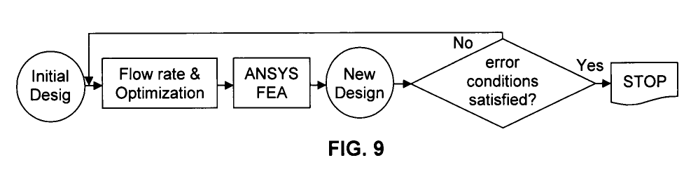

The finite element analysis (FEA) together with the membrane optimization algorithm and flow rate analysis to characterize the structural behavior of the entire systolic nanopump will be performed. As shown in FIG. 9, the optimized membrane dimensions and dimensions of micro-valve are provided as inputs for ANSYS FEA package. The iterative procedures are required to find the optimized structure of the systolic micro pump to fulfill all requirements predetermined by the constraints and side constraints. The membrane and systolic micro pump will be modeled and meshed in 10-node tetrahedron elements for complete 3-D structural analysis and simulation.

### Identification of NEMS Fabrication Processes

To be better prepared for the fabrication of the systolic nanopump, the NEMS fabrication techniques will be identified appropriately to package the designed systolic nanopump. The selection criteria set forth for specific medical applications are:

1), the material used must be biocompatible,
2), according to our designed dimensions, larger ranges of single layer thickness and minimum feature width are needed,
3), since sidewall is to be structured for the micro-chamber, micro fabrication with high aspect ratio is required,
4), higher productivity and lower cost.

In vitro Experimental Study

The scaled up systolic micro pump (with diameter about 1 cm) will be constructed for bench-top testing and validation in an elevated SBP chamber. Since the systolic micro pump is self-actuated at a designated SBP level, this study is crucial to understand the efficacy and functionality of the systolic micro pump in response to pressure elevation for hypertension therapy.

A SBP chamber will be constructed (6-in long using ¾-inch thick clear Acrylic such as Lucite® or Acrylite®) to simulate the SBP elevation in hypertension patients. The use of transparent material will enable the researcher to visually observe the fluid flow condition inside the SBP chamber if the pumped fluid is dyed. The SBP chamber in the simplified cylindrical shape (with diameter 6-in) will be constructed. The SBP chamber will be composed of two segments; these two segments can be fastened and sealed together without any pressure leakage during experiment. The two-unit design and augmentation of the SBP chamber make it easier to place and retrieve the systolic micro pump from the SBP chamber.

During the experiment, the central circle of the SBP chamber will be divided into a 2-D cylindrical grid system. The systolic micro pump will be placed at different nodal position of the grid system to quantify its drug delivery rate at different locations inside the SBP chamber; that is, inside the blood vessel. The protocol is as follows:
1) Situate the systolic nanopump, which carries a known amount of drug concentration inside the micro-chamber, at a nodal position.
2) Fill the SBP chamber with transparent liquid that simulates blood.
3) Tightly fasten and seal the two units.
4) Elevate the pressure inside the SBP chamber to simulate the rise of SBP. Pressure elevation at the experimental node will be monitored by a pressure sensor.
5) Once the pressure reaches the designated SBP level, the pressure will be released from the SBP chamber via a two-way valve.
6) The SBP chamber will then be opened and the systolic nanopump is retrieved to measure the amount of liquid (q′) that has been pumped out of the systolic nanopump.
7) The experiment will be repeated for different nodal positions and SBP levels.
8) Drug-delivery rate (DDR) of the systolic nanopump is defined as

$$DDR = \frac{{q}^{′}}{q} \qquad ( 6 )$$

where q is the analytical value (100% delivery rate) of the drug-fluid to be discharged from the systolic nanopump. This empirical study will map the DDR in the blood vessel, which is important to identify the location at which the maximum DDR occurs. Findings from this study will be helpful to our Phase II animal and human subject studies where the systolic nanopump should be implanted. It will also improve the efficacy of drug delivery for hypertension treatment.

Finally, it is understood that the invention is not limited to the disclosed structures, devices, materials, elements, methods, applications and embodiments shown, including any embodiments that may be apparent to one of ordinary skill in the art. Although the foregoing invention has been described in some detail by way of illustration for purposes of clarity of understanding, it will be readily apparent to those of ordinary skill in the art in light of the teachings of this invention that certain variations and modifications may be made thereto without departing from the spirit or scope of the disclosure herein, including the specific embodiments. In other words, numerous other variations of the present invention will be appreciated by those skilled in the art in view of the disclosure herein.

What is claimed is:

1. A nano pump implantable in the body for delivering drugs or active agents, said nano pump comprising:
a) an inlet channel;
b) an inlet nano-valve;
c) a chamber for holding said drugs or active agents;
d) a membrane for pumping said drugs or active agents, wherein the membrane is designed to deflect in response to rise in blood pressure, blood lipid or blood glucose to a designated level, thereby pumping out the drugs or active agents without any other input source;
e) an outlet nano-valve; and
f) an outlet channel.

2. The nano pump of claim 1, wherein the thickness of the membrane is determined based on the designated level of blood pressure, blood lipid or blood glucose at which the drugs or active agents have to be pumped out.

3. The nano pump of claim 1, wherein the membrane includes a membrane pillar.

4. The nano pump of claim 1, wherein the membrane has a circular design.

5. The nano pump of claim 1 linked to similar nano pumps to form a linked array or a 2-D matrix of nanopumps for pumping larger doses.

6. The nano pump of claim 1 linked to similar nano pumps which have different membrane thickness which are designed for multi-level drug release.

7. The nano pump of claim 1, wherein the material, thickness, and diameter of the membrane is designed to compensate for the viscosity effect in the nano pump.

8. The nano pump of claim 1, wherein the nano-valves are composed of two parts.

9. An implantable nano pump for drug delivery, wherein said nano pump consists essentially of:
a) an inlet channel;
b) an inlet nano-valve;
c) a chamber for holding said drug;
d) a membrane for pumping said drug, wherein the membrane is designed to deflect in response to rise in blood pressure, blood lipid or blood glucose to a designated level, thereby pumping out the drug without any another input source;
e) an outlet nano-valve; and
f) an outlet channel.

10. The nano pump of claim 9, wherein the membrane includes a membrane pillar.

11. The nano pump of claim 9, wherein the membrane has a circular design.

12. The nano pump of claim 9 linked to similar nano pumps to form a linked array or a 2-D matrix of nanopumps for pumping larger doses.

13. The nano pump of claim 9 linked to similar nano pumps which have different membrane thickness which are designed for multi-level drug release.

14. The nano pump of claim 9, wherein the material, thickness, and diameter of the membrane is designed to compensate for the viscosity effect in the nano pump.

15. An implantable nano pump for drug delivery, wherein said nano pump consists of:
a) an inlet channel;
b) an inlet nano-valve;
c) a chamber for holding said drug;
d) a circular membrane for pumping said drug, wherein the membrane includes a membrane pillar is designed to deflect in response to rise in blood pressure, blood lipid or blood glucose to a designated level, thereby pumping out the drug without any another input source;
e) an outlet nano-valve; and
f) an outlet channel.

16. The nano pump of claim 15 linked to similar nano pumps to form a linked array or a 2-D matrix of nanopumps for pumping larger doses.

17. The nano pump of claim 15 linked to similar nano pumps which have different membrane thickness which are designed for multi-level drug release.

18. The nano pump of claim 15, wherein the material, thickness, and diameter of the membrane is designed to compensate for the viscosity effect in the nano pump.
